(19) United States
(12) Patent Application Publication
(10) Pub. No.: US 2015/0344399 A1
(43) Pub. Date: Dec. 3, 2015

(54) Heat as a Method to Enhance the Fluid Activating Ability of Humic Acids, Zeolites and related Enerceuticals

(71) Applicant: William John Martin, South Pasadena, CA (US)

(72) Inventor: William John Martin, South Pasadena, CA (US)

(21) Appl. No.: 14/294,076

(22) Filed: Jun. 2, 2014

(51) Int. Cl. C07C 65/105

(52) U.S. Cl. CPC C07C 65/105

(57) ABSTRACT

The invention relates to the use of heat and/or acids to increase the water activating property of various substances, including humic acids and zeolites. The basic theme is that, whenever possible, water being consumed by humans and animals, or being used in agriculture, should be pre-activated using one or other of the available methods. Humic acids and zeolites have been shown to have water-activating activities. Pre-heating these materials to temperatures in the range of 1,000° C., significantly increases this water-activating activity. Lesser increases in water activation are achievable using 200° C. heat and/or exposure to low pH using acids. Other fluids, including alcoholic beverages, can similarly be more efficiently activated using heated versus unheated humic acids and/or zeolites. This discovery will help facilitate the utilization of activated water in various fields, including human and animal consumption, agriculture and industry.

### CROSS REFERENCE TO RELATED APPLICATIONS

Co-Pending Patent Applications:

Methods for detection of ultraviolet light reactive alternative cellular energy pigments (ACE pigments). William John Martin Submitted Dec. 24, 2007. Publication number 20090163831

Method of assessing and of activating the alternative cellular energy (ACE) pathway in the therapy of diseases. William John Martin Submitted Jan. 16, 2008. Publication number 20090181467

Enerceutical mediated activation of the alternative cellular energy (ACE) pathway in the therapy of diseases. Submitted May 8, 2008. Publication number 20090280193

Regenerative wound healing using copper-silver citrate composition. Submitted Oct. 22, 2008 Publication number: 20100099758.

Enerceutical activation of the alternative cellular energy (ACE) pathway in therapy of diseases. Submitted Feb. 11, 2009. Publication number 20090202442.

Method of using the body's alternative cellular energy pigments (ACE-pigments) in the therapy of diseases Submitted Feb. 20, 2009. Publication number 20100215763

Urine as a source of alternative cellular energy pigments (ACE-pigments) in the assessment and therapy of diseases. Submitted Mar. 5, 2009. Publication number 20100196297

Moringa oil mediated activation of the alternative cellular energy pathway in the therapy of diseases. Submitted Feb. 24, 2010. Publication number 20110208110.

Activation of the alternative cellular energy (ACE) pathway in the therapy of diseases. Submitted Jun. 9, 2010. Publication number 20110306917.

Methods for the detection of alternative cellular energy (ACE) pigments and for monitoring of the ACE pathway in the diagnosis and therapy of diseases. Submitted Jun. 13, 2010. Publication number 20110306077.

Diagnostic value of systemic ACE pathway activation in the detection by fluorescence of localized pathological lesions. Submitted Jul. 26, 2010. Publication number 20100291000

Enerceutical mediated activation of the alternative cellular energy (ACE) pathway in the therapy of diseases. Submitted July 2010.

Method of generating hydrogen in gasoline using an enerceutical product added to magnesium in a hydrogen permeable but solute impermeable container. Submitted Jul. 18, 2008. Publication number 20100011657

Energy Charged Liquids to Enhance Enerceutical Activation of the Alternative Cellular Energy (ACE) Pathway in the Therapy of Diseases. Submitted Dec. 17, 2010. Publication number 20120152755

Energy Charged Alcoholic Beverages for Enhancing the Alternative Cellular Energy Pathway in the Prevention and Therapy of Diseases. Submitted January 2011, Publication number 20120171340.

Methods for Detecting and Monitoring the Activity of Energized Water and Other Liquids Useful for Enhancing the Alternative Cellular Energy Pathway in the Prevention and Therapy of Diseases. Submitted February 2011

Methods for Increasing the Kinetic Activity of Alcohol, Water and Other Liquids, so as to Render the Liquids More Useful in Enhancing the Alternative Cellular Energy Pathway in the Prevention and Therapy of Diseases. Submitted February 2011

Methods for Increasing the Kinetic Activity of Water and Other Liquids, so as to Render the Liquids More Useful in Enhancing the Alternative Cellular Energy Pathway and in Various Other Agricultural and Industrial Applications. Submitted June 2011.

Methods for Increasing the Kinetic Activity of Water and Other Liquids, so as to Render the Liquids More Useful in Enhancing the Alternative Cellular Energy Pathway and in Various Other Agricultural and Industrial Applications. Submitted October 2011.

Use of Plants Extracts to Activate Water, Alcohol and Other Liquids. Submitted Oct. 27, 2011. Application Ser. No. 13/272,215.

Methods of Transferring Energies to Water, Alcohols and Minerals. Submitted Nov. 25, 2011. Application Ser. No. 13/304,558.

Weight Change as a Measurement of an Intrinsic Energy Property of Matter. Submitted Dec. 27, 2011. Application Ser. No. 13/340,669

Weight Change as a Measurement of an Intrinsic Energy Property of Foods and Other Materials, Submitted Jan. 5, 2012.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not Applicable

### BACKGROUND OF THE INVENTION

As detailed in earlier co-pending patent applications, the inventor has linked an inducible kinetic activity of activated water to the water's capacity to provide biological benefits beyond those provided by regular, non-activated water. Water activation, in terms of the water acquiring KELEA™ (kinetic activity limiting electrostatic attraction), can be achieved using a variety of methods, including the addition to the water of humic/fulvic acids, zeolites and various ceramics. Activation can be assessed by the differing dissolving patterns of particles of neutral red dye sprinkled onto the surface of the water. These patterns can range from stationary particles with slowly enlarging concentric rings of dissolved dye (indicative of minimal kinetic activity of the water), to rapid linear movements of the particles in activated water. The linear movements have a to-and-fro quality and can lead to long streaks of dissolved dye. In highly activated water, the particles can even move as a rapid horizontal vortex. While dissolved particles of neutral red dye in regular water, do not yield an ultraviolet (UV) light fluorescent solution, activated solutions with added neutral red dye will fluoresce upon UV illumination. Another useful assay to assess the degree of kinetic activation of water is to measure the rate of weight loss of capped containers of the water. For non-activated water, the weight reduction even over several hours is minimal (<0.1 mg per ml of water). Activated water, will lose weight, primarily due to evaporation, more rapidly (>0.5 mg/ml), even reaching >5 mg/ml). The increased vaporization is essentially a measure of reduced intermolecular hydrogen bonding of the water molecules and can also be also measured as an increase in vapor pressure. Activation can be shown with other fluids, including alcohol and alcoholic beverages, which have a higher baseline of activity than water, but can also be further induced to much higher levels of activity. It has also been noted that a small quantity of activated fluid added to regular fluid will induce activation of the entire fluid in a time dependent manner. This type of progressive activation, readily achieved using 10 fold dilutions of activated water into regular water, is similar to the method used in preparing, so called, homeopathic formulations. Unlike, the misleading principle that homeopathic formulations have a specificity of action under the “law of similars,” activated water and/or alcohol can potentially provide substantial clinical benefits for a wide range of illnesses. The clinical benefits occur through the activation of body's alternative cellular energy (ACE) pathway upon the consumption, injection or even being in close proximity of the activated fluid.

This new medical paradigm has placed special importance on increasing the efficiency of water activation. It was reasoned that products like humic acid and zeolites were able to absorb KELEA energy from the environment because of their dielectric (electrical charge polarization) characteristic. The working hypothesis is that KELEA is attracted by free (unbound) electrical charges and may be fundamental in preventing the fusion and possible annihilation of opposing electrical charges. For certain molecules, including humic acids and zeolites, the free charges are reflected in their electrostatic property, especially to the extent that the separated electrical charges can remain free from covalent and to a lesser extent from hydrogen bonding. It is envisioned that these dielectric compounds can capture and then release (transmit) KELEA in an oscillating manner, such that nearby dielectric molecules will also undergo a further charge separation and potentially become direct receivers of KELEA. Based on this premise and on other clues from empirical observations, it was reasoned that heating and/or acid treatment of humic acids and zeolites will lead to the breaking of some of the covalent bonds and hydrogen bonds, thereby, rendering these materials more effective in capturing and releasing KELEA. From a practical point of view, these treatments should enhance the water and alcohol activation ability of these products. All patent applications listed in this specification are herein incorporated by reference.

### BRIEF SUMMARY OF THE INVENTION

The water activating activity of humic acids and zeolites can be significantly enhanced by heating the materials to high temperatures (˜1,000° C.) or by prolonged strong acid treatment (pH<2).

### BRIEF DESCRIPTION OF THE DRAWINGS

Not Applicable and none included

### DETAILED DESCRIPTION OF THE INVENTION

Samples of humic acid were subjected to heating at different temperatures (200° C. and 1,000° C.) and tested in assays for water and alcoholic beverage activating activity. The water tested was both tap water and bottled water, purchased at a Safeway Store (Refreshe). The alcoholic beverages tested included EverClear (75% ethanol) and Stroh's Rum (80% ethanol). The source of humic acid was Moringstar Minerals, Farmington, N. Mex., while the zeolite was mined in Japan. It comprised 76% silicon oxide, 11.5% aluminum oxide, 5.2% potassium oxide, 3.0% ferrous oxide, 2.1% calcium oxide, 1.4% sodium oxide and trace amounts of other oxides. A regular air over was used to reach 200° C., which was maintained for 4 hours. A vacuum oven was used for the 1,000° C. heating (Aremac, City of Industry, Calif.), and maintained at this temperature for 1 hour. Unheated humic acids and zeolites were used as controls.

Humic acids and zeolites heated to 1,000° C. performed far better than unheated control materials in the activation of water and alcoholic beverages. The enhanced activation was seen microscopically in the continuing movement of fine particles of the materials, simply suspended in the fluids. It was also seen in the more dramatic movements and dissolving patterns of neutral red dye sprinkled onto small plastic dishes containing water plus the heated materials, when compared with the use of control materials. In a weight loss experiment, ˜1 mg/ml in of heated humic acid in water led to a reduction of weight of 0.90 mg/ml over a 12 hour period compared to a negligible 0.02 mg/ml using the same very small amount of humic acid. (larger quantities of unheated humic acid regularly induce significant weight loss when tested in this manner). For the heated and unheated zeolites, the corresponding values were 1.86 mg/ml and 0.57 mg/ml, respectively. Heating the materials to 250° C. for 4 hours saw less impressive, but still discernable, increases in the water activating activity of humic acids and zeolites. Heating to 1,000° C. for only 1 hour, however, achieved a markedly greater level of enhanced water-activating activity, making it a suitable method for further studies.

Humic acids and zeolites samples were also tested for water activating ability after being added for a day to concentrated hydrochloric acid (37.5%). The acid was removed by washing in water and the materials tested for water activating properties compared to untreated materials. Based on the induced heightened dissolving pattern of neutral red dye, water exposed to acid treated humic acids and zeolites showed heightened activity, although less than achieved by the 1,000° C. heating.

The primary purpose of this application is, therefore, to record the quite striking benefit of using humic acids and zeolites heated to 1,000° C. Vacuum ovens, which can heat up to 3,000° C. are available and will undoubtedly result in more covalent bond breakage than 1,000° C.

Humic acids are a widely accepted dietary supplement. At least for humans, it is preferable to zeolites because of the possible toxicity of the silica and aluminum in zeolites, as opposed to the carbon based humic acids. In certain applications, described in co-pending patent applications, the energized solutions can be used externally, without any direct contact with the individual being treated. In one embodiment, neutral red dye in an activated solution is placed into a plastic Ziploc bag. The bag is placed onto the skin, for example over a site of active or recurrent herpes simplex virus (HSV) infection, or an area of skin with underlying collection of alternative cellular energy (ACE) pigments. The bag is illuminated with ultraviolet (UV) light in a manner that induces activation of the body's ACE pathway, as shown by the development of direct UV inducible fluorescence of the underlying skin. Containers of highly activated solutions, either with or without addition of neutral red dye and UV illumination, can also lead to the activation of regular water placed in the proximity to the activated solution. Activated solutions are not dependent upon the continuing presence of the humic acids and zeolites and the activating materials can be removed from the activated solutions. Following activation of solutions, the residual materials can be removed by simple Millipore or other filtration methods or by centrifugation. Removal of the heated materials from activated solutions will facilitate regulatory approvals for the widespread human consumption of the solutions, as envisioned in upcoming planned clinical trials. The amounts of heated humic acids and/or zeolites required to activate water and/or alcoholic beverages are relatively modest. Ten to 100 mg/ml of heated humic acids are quite suitable and can be repeatedly used to activate further fluid samples. Lower amounts are required if sufficient time is allowed for fluid activation to occur. Activation is more efficient and long-lasting using sealed containers. Once activated, the fluids can be used to further activate additional quantities of fluids.

Both humic acids and zeolites have widespread use in agriculture, as soil amendments and as direct stimulants of the growth and vitality of crops. These products are also generally useful in being able to reduce dependency on the use of conventional fertilizers and pesticides. While generally considered to be beneficial because they provide bio-available minerals, I have concluded that a major benefit of humic acids and zeolites in human, animal and agricultural applications is their ability to activate water. The invention described, herein, is that this important property of humic acids and zeolites can be improved by heating of the materials before use. Indeed, it can now be argued that essentially all of the water being used in agriculture should be activated. The invention provides a means of efficiently achieving this goal.

The KELEA activated fluids have been shown to have several major industrial applications, referred to in prior and co-pending patent applications. These include the more efficient combustion of gasoline, suppression of scale formation in water-cooled heat exchangers, improved and more even dispersion of cements, paints and other colloidal suspensions. KELEA is also likely to reverse the upward seepage of groundwater into the foundations and lower walls of old buildings. Of recent interest is the apparent capacity of KELEA to reduce resistance to the flow of electricity within electrical circuits. In all of these applications, the availability of a more potent transmitter of KELEA, as can be obtained using heated humic acids and zeolites, when compared to unheated control materials, will lead to greater efficiency and assist in gaining an increased scientific understanding of the underlying physics.

Although the focus of this application has been on humic acids and zeolites, the basic understanding of increasing the water activation of materials, using heat and or acid treatment, will likely have more general applications. In particular, the methods are testable with other products, which can be used to achieve a level of water activation. These include mica; kaolin (edible clay); various crystals; shungite and other fullerenes; extracts of plants, such as moringa oleifera and ashitaba; etc. The clinical beneficial activities of these natural products have been ascribed to their capacity of enhancing the ACE pathway. For this reason, the products have been termed enerceuticals™. This concept has now been further refined as their ability to activate water, which can clearly include the body's intracellular and extracellular fluids. The concept also applies to enhancing the kinetic and biological activities of the fluid components of plants.

The invention now being described, it will be apparent to one of skill in the art that many changes and modifications can be made thereto without departing from the spirit and scope of the appended claims.

1. A method of enhancing the fluid activating function of humic acids, comprising the heating of humic acids to a temperature in the range of 1,000° C. for sufficient time that when a sample of the heated humic acids is added to water and/or alcohol containing solutions it results in the solutions acquiring more kinetic activity than shown by comparable solutions to which an equivalent amount of unheated humic acids is added.

2. A method of enhancing the fluid activating function of humic acids, comprising the heating of humic acids to a temperature in the range of 1,000° C. for sufficient time that when a sample of the heated humic acids is added to water and/or alcohol containing solutions it results in the solutions acquiring more kinetic activity than shown by comparable solutions to which an equivalent amount of unheated humic acids is added.

3. A method of enhancing the fluid activating function of humic acids and/or zeolites, comprising the exposure of these substances to acid for sufficient time that when a sample of the acid treated humic acids is added to water and/or alcohol containing solutions it results in the solutions acquiring more kinetic activity than shown by comparable solutions to which an equivalent amount of untreated humic acids and/or zeolites is added.

4. The method of claim 1, in which following the fluid activation process, the residual heated humic acids are removed from the activated fluid by filtration and/or by centrifugation.

5. The method of claim 2, in which following the fluid activation process, the residual heated zeolites are removed from the activated fluid by filtration and/or by centrifugation.

6. The method of claim 1 in which the fluid is gasoline, with the intention of using the heated humic acids to increase the vaporization and fuel efficiency of the gasoline upon its combustion.

7. The method of claim 2 in which the fluid is gasoline, with the intention of using the heated zeolites to increase the vaporization and fuel efficiency of the gasoline upon its combustion.

8. The method of claim 1 in which the activated fluid is used to provide a more effective source of transmitted KELEA (kinetic energy limiting electrostatic attraction) than provided by fluid in which a comparable amount of unheated humic acids was added.

9. The method of claim 2 in which the activated fluid is used to provide a more effective source of transmitted KELEA (kinetic energy limiting electrostatic attraction) than provided by fluid in which a comparable amount of unheated zeolites was added.

10. The method of claim 1 in which, rather than applying heat to humic acids, it is applied to other types of materials, which are known for their ability to activate water in a manner similar to that achievable by using humic acids.
